(19) United States
(12) Patent Application Publication
(10) Pub. No.: US 2017/0360613 A1
(43) Pub. Date: Dec. 21, 2017

(54) Treatment Device and System

(71) Applicant: CEL Tech, LLC, Hermosa Beach, CA (US)

(72) Inventors: Louis Shoch, Hermosa Beach, CA (US); Eddie Becdach, Hermosa Beach, CA (US); Chris Spanellis, Hermosa Beach, CA (US)

(21) Appl. No.: 15/625,917

(22) Filed: Jun. 16, 2017

Related U.S. Application Data
(60) Provisional application No. 62/351,883, filed on Jun. 17, 2016.

(51) Int. Cl. A61F 11/06; A61F 7/02; A61H 1/00; A61F 7/00

(52) U.S. Cl. CPC A61F 11/06; A61H 1/006; A61F 7/02; A61F 2007/0005; A61F 2007/0225

(57) ABSTRACT

A treatment system including a treatment device for treating a treatment area of a person's body is provided. The treatment device may include one or more treatment elements or treatment balls and a clamp or securement mechanism. The clamp or securement mechanism may include a first opposable portion and a second opposable portion, wherein at least one of the first opposable portion and the second opposable portion includes a receptacle configured to releasably hold a treatment element. The clamp or securement mechanism may be transitioned between an open configuration and a closed configuration. The treatment device may be used to apply pressure and a desired temperature to the treatment area, such as to apply pressure and a cold temperature to a portion of a person's ear to help treat ear injuries, which may help to reduce the likelihood of developing deformities (e.g., “cauliflower ear”).

### PRIORITY

This application claims priority to U.S. Provisional Application No. 62/351,883, filed Jun. 17, 2016, which is incorporated by reference in its entirety into this application.

### BACKGROUND

Various parts of a person's body may receive injuries for various reasons. These injuries may require treatment. For example, boxing, wrestling, martial arts, mixed martial arts (MMA) and other contact sports and activities can result in damage to a person's ear. If not properly treated after injury, damage to a person's ear may heal improperly or may result in deformities in the ear. These deformities are sometimes referred to as “cauliflower ear.” Blunt force injuries to the ear can lead to a blockage that prevents blood flow and damages tissue. This can result in a bumpy or lumpy appearance on part of the ear, similar to a cauliflower. Early treatment can help prevent permanent deformity.

Disclosed herein are new devices, components, assemblies, systems, and methods for early and effective treatment of injuries (e.g., injuries to the ear), which avoid disadvantages, help prevent swelling, and promote healing. These may provide treatment by applying consistent pressure and a desired temperature to the injured area. For example, if used to treat an injury to an ear with pressure and a cold temperature, these may help prevent formation of deformities on the ear and provide other benefits as described herein. The concepts described herein may be applied to a wide variety of treatments, therapies, and uses.

### SUMMARY OF THE INVENTION

Embodiments of, and enhancements for, apparatuses, devices, components, assemblies, systems, methods, etc. for ear treatment devices and for preventing deformities of the ear, are described herein. A variety of embodiments are encompassed, but a few non-limiting examples are described in more detail.

In one embodiment, a treatment system and/or treatment device (e.g., an ear treatment system or device) for treating a treatment area of a person's body (e.g., an injured portion of a person's ear) may include one or more treatment elements (e.g., a first treatment element or treatment ball similar to any described in this disclosure) and a clamp or clamping/securement mechanism (e.g., the same as or similar to any described in this disclosure). The clamp or clamping/securement mechanism may include a first opposable portion and a second opposable portion (e.g., the same as or similar to the opposable portions described in this disclosure), wherein the first opposable portion includes a first receptacle configured to releasably hold the treatment element, and wherein the securement mechanism is transitionable between one or more open configurations and closed configurations. The closed configuration(s) may have a distance between the first opposable portion and the second opposable portion (e.g., a distance between a first point on the first opposable portion and a second point on the second opposable portion) that is less than the distance therebetween in the open configuration(s). The closed configuration may be designed/configured such that it can clamp/hold a portion of a person's body (e.g., a portion of an ear) between the first opposable portion and the second opposable portion such that pressure is applied to the portion of the person's body. The second opposable portion may include a platform configured to apply an opposing force to the treatment element when in the closed configuration. The second opposable portion may include a second receptacle capable of receiving a second treatment element. The clamp or clamping/securement mechanism may be configured such that the distance between the first opposable portion and the second opposable portion can be adjusted between 2-10 (e.g., 3) closed configurations that each apply a different amount of pressure to the treatment area

The treatment element(s) may be a variety of shapes and sizes, e.g., the treatment element(s) may be formed as a treatment ball(s) or may be spherical or partially spherical. The treatment element(s) may include an outer wall/shell and includes at least one of a gas, liquid, and gel inside the outer shell. The treatment element may be configured such that it can be cooled to a temperature below room temperature and remain below room temperature for a period of time between 2-30 minutes, 3-20 minutes, or longer than 4 minutes. The treatment element may be configured such that it can be heated to a temperature above room temperature and remain above room temperature for a period of time between 2-30 minutes, 3-20 minutes, longer than 4 minutes, or longer than 8 minutes. The treatment element(s) may include an outer surface and may be flexible enough to change a shape of the surface when pressure is applied to the treatment area with the treatment element.

In one embodiment a method (e.g., a method of treating a treatment area of a person's body or ear) may include obtaining a treatment device (e.g., a treatment device for treating a treatment area of a person's body). The treatment device (e.g., an ear treatment device) may include one or more treatment elements (e.g., a first treatment element and/or 1-25 treatment elements) and a clamp or clamping/securement mechanism. The treatment element(s) may be the same as or similar to any of the treatment elements described in this disclosure. The clamp or clamping/securement mechanism may be the same as or similar to any of the clamp or clamping/securement mechanisms described in this disclosure. The clamp or clamping/securement mechanism may include a first opposable portion and a second opposable portion (e.g., the same as or similar to the opposable portions described in this disclosure). One or both of the first opposable portion and the second opposable portion may include one or more receptacle(s) configured to releasably hold a treatment element. The securement mechanism may be transitioned between an open configuration and a closed configuration, e.g., similar to the open configurations and closed configurations described elsewhere herein.

The method may also include altering a temperature of the treatment element(s) from a first temperature to a second temperature different from the first temperature. The second temperature may be colder or hotter than the first temperature. Altering the temperature of the treatment element(s) from the first temperature to the second temperature may include cooling the treatment element(s) (e.g., in a refrigerator or a freezer). Altering the temperature of the treatment element(s) from the first temperature to the second temperature may include heating the treatment element(s) (e.g., in hot water, on a stove, in an oven, etc.). The method may also include adjusting the temperature of the treatment element(s) multiple times (e.g., a second, third, and/or fourth time). Each of these temperatures may be the same or different, e.g., this may involve altering a third temperature of the treatment element(s) to a fourth temperature of the treatment element, the fourth temperature being different from the third temperature.

The method may also include placing a treatment element in a receptacle such that it is held by the first opposable portion or the second opposable portion, or placing treatment elements in receptacles in each of the first opposable portion and the second opposable portion. The method may also include securing the treatment device at the treatment area by transitioning the treatment device from the open configuration to the closed configuration with the treatment area positioned/located between the first opposable portion and the second opposable portion and/or between treatment elements or a treatment element and an anvil/platform. This can be done such that, in the closed configuration, the treatment element(s) applies pressure to the treatment area and applies the second temperature or another temperature to the treatment area. The method may also include removing the treatment device from the treatment area after a period of time. The method may also include applying pressure to the treatment area with the treatment element(s) for a period of time (e.g., between 1-30 minutes or 2-15 minutes) before removing the treatment device from the treatment area.

The method may also include removing the treatment element(s) from the receptacle(s) after the initial treatment period of time. The method may also include altering a third temperature of the treatment element(s) to a fourth temperature of the treatment element(s) after removing the treatment element(s) from the receptacle(s), the fourth temperature being different from the third temperature. The method may also include then returning the treatment element(s) to the receptacle(s) such that the treatment element(s) is held by the first opposable portion and/or second opposable portion. The method may also include resecuring the treatment device at the treatment area such that the treatment element(s) applies pressure to the treatment area after returning the treatment element(s) to the receptacle(s).

The method may also include obtaining one or more new treatment element(s) different from the initial treatment element(s), and placing the one or more new treatment element(s) in the one or more receptacle(s) such that the new treatment element(s) are held by the first opposable portion and/or the second opposable portion. The method may include then resecuring the treatment device at the treatment area such that the new treatment element(s) applies pressure to the treatment area.

The securement mechanism may include a lock/release lever (e.g., one that has teeth), and securing and/or resecuring the treatment device at the treatment area may include adjusting a distance between the first opposable portion and the second opposable portion until the teeth lock the first opposable portion and the second opposable portion in a first position that applies a first pressure to the treatment area. Securing and/or resecuring the treatment device at the treatment area may also include readjusting the distance between the first opposable portion and the second opposable portion until the teeth lock the first opposable portion and the second opposable portion in a second position that applies a second pressure to the treatment area, wherein the second pressure is different from the first pressure. This may also include readjusting the distance between the first opposable portion and the second opposable portion until the teeth lock the first opposable portion and the second opposable portion in a third position that applies a third pressure to the treatment area, wherein the third pressure is different from the first pressure and the second pressure. Removing the treatment device from the treatment area may include adjusting the lock/release lever such that the first opposable portion and the second opposable portion are released from the first position, second position, third position, or another position such that the treatment device is able to transition to the open configuration and/or a different position applying less pressure.

In one embodiment, treatment systems, treatment devices, ear treatment systems/devices, apparatuses, components, systems, etc. may include any features described herein, depicted in the figures, or known in the art. And these may be used in various treatment methods including any combination of the various steps described herein and any steps known in the art.

### BRIEF DESCRIPTION OF THE DRAWINGS

The disclosed devices, apparatuses, components, assemblies, systems, methods, etc. can be better understood with reference to the description taken in conjunction with the following drawings, in which like reference numerals identify like elements. The components in the drawings are not necessarily to scale.

FIG. 1 shows a perspective view of an exemplary treatment device in an open configuration;

FIG. 2 shows a side view of the treatment device of FIG. 1 in a closed or clamped configuration;

FIG. 3A shows an exemplary embodiment of a treatment element that may be used with the treatment device of FIG. 1;

FIG. 3B shows a side view of an exemplary embodiment of a treatment element that may be used with the treatment device of FIG. 1, wherein the treatment element has been cut in half to show its interior;

FIG. 4 shows an exemplary embodiment of an upper clamp portion of a clamp or clamping/securement mechanism that may be used in the treatment device of FIG. 1;

FIG. 5 shows an exemplary embodiment of a lower clamp portion of a clamp or clamping/securement mechanism that may be used in the treatment device of FIG. 1;

FIG. 6 shows an exemplary embodiment of a lock/release of a clamp or clamping/securement mechanism that may be used in the treatment device of FIG. 1.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Described herein are devices, components, assemblies, systems, methods, etc. for treatment devices and systems (e.g., ear treatment devices and systems). The description and accompanying figures, which describe and show certain embodiments, are made to demonstrate, in a non-limiting manner, several possible configurations of treatment devices, apparatuses, devices, components, assemblies, systems, etc. and various methods of using them according to various aspects and features of the present disclosure. Accordingly, the disclosure is not limited to the specific embodiments described. Rather, the inventive principles associated with the embodiments described herein, including with respect to the apparatuses, devices, components, assemblies, systems, methods, etc. described herein, may be applied in a variety of ways, including to other types of apparatuses, devices, components, assemblies, systems, methods, etc. General and specific apparatuses, devices, components, assemblies, systems, methods, etc. are described herein sufficiently to enable one to develop a variety of implementations/applications without undue experimentation. In the development of particular applications, numerous implementation-specific decisions will be made to achieve the design-specific goals, which will vary from one implementation/application to another. It will be appreciated that, having access to this disclosure and reading this disclosure, such a development effort would be a routine undertaking for persons of ordinary skill in the art.

This document does not intend to distinguish between components that differ in name but not function. In the following discussion and in the claims, the terms “including,” “includes,” “comprising,” “have,” and “has” are used in an open-ended fashion, and thus should be interpreted to mean “including, but not limited to.” The word “or” is used in the inclusive sense (i.e., “and/or”) unless a specific use to the contrary is explicitly stated.

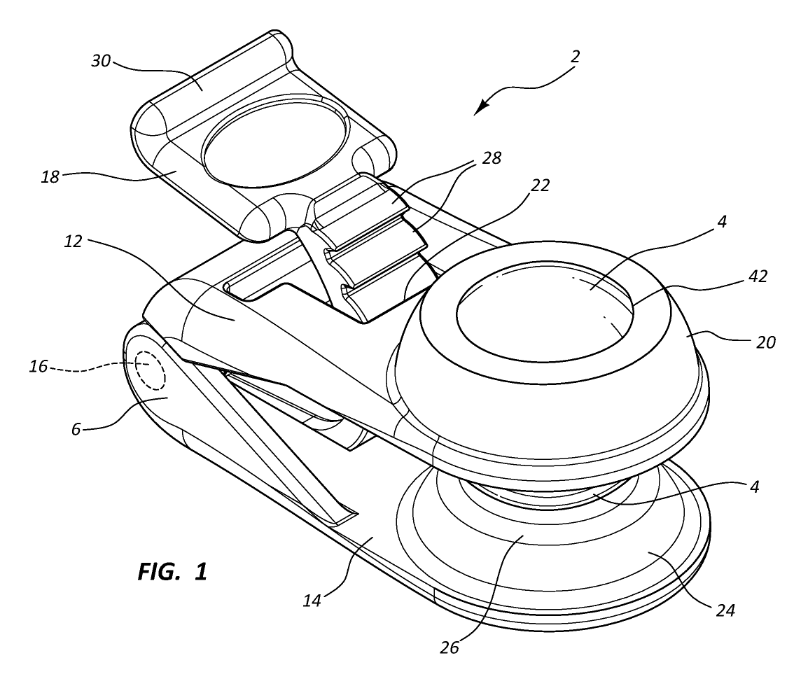

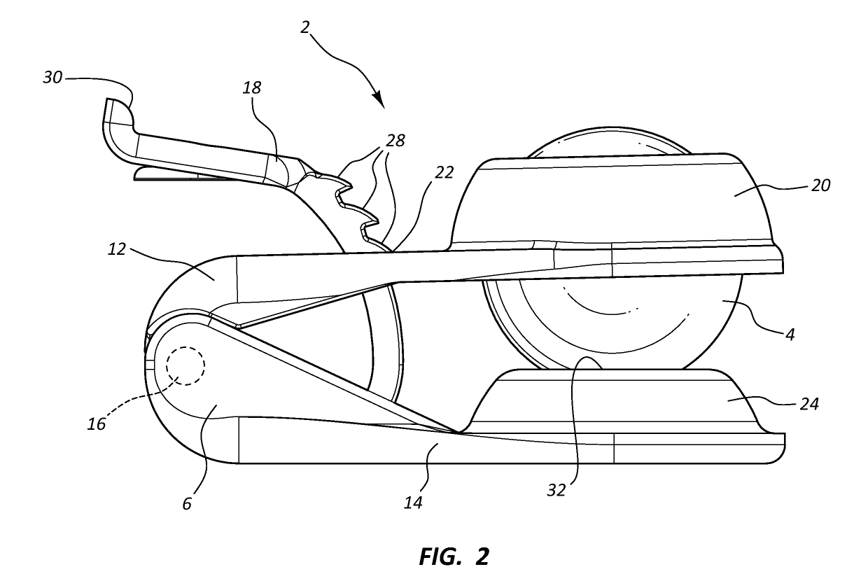

FIG. 1 shows a perspective view of an exemplary treatment device 2 in an open configuration. FIG. 2 shows a side view of the treatment device 2 of FIG. 1 in a closed or clamped configuration. The treatment device 2 may include one or more treatment elements 4 and a clamping/securement mechanism 6 (e.g., a clamp). The treatment device 2 may be used to apply pressure and/or a temperature(s) (e.g., a cold or cool temperature(s)) to a portion of person's body to be treated. In one embodiment, the treatment device 2 is configured/designed as an ear treatment device that may be used to treat an injured portion of a person's ear, e.g., to apply pressure and cold to an ear injury to help prevent cauliflower ear from forming. The clamping/securement mechanism 6 may act to hold the treatment element(s) 4 against a treatment area (i.e., a portion of a person's body to be treated) and apply pressure to the treatment area. The treatment element(s) 4 may be cooled or include a cooling gel or other material, such that the treatment ball can apply cold to the treatment area. For example, if a person injures his/her ear during boxing, wrestling, mixed martial arts, etc., the person may apply the treatment device 2 as an ear treatment device to the injury or the treatment area (e.g., the portion of the ear that includes the injury) to apply pressure and/or cold temperature to the ear and, thereby, treat the injury. This treatment may reduce the risk of the person developing deformities in the ear or cauliflower ear.

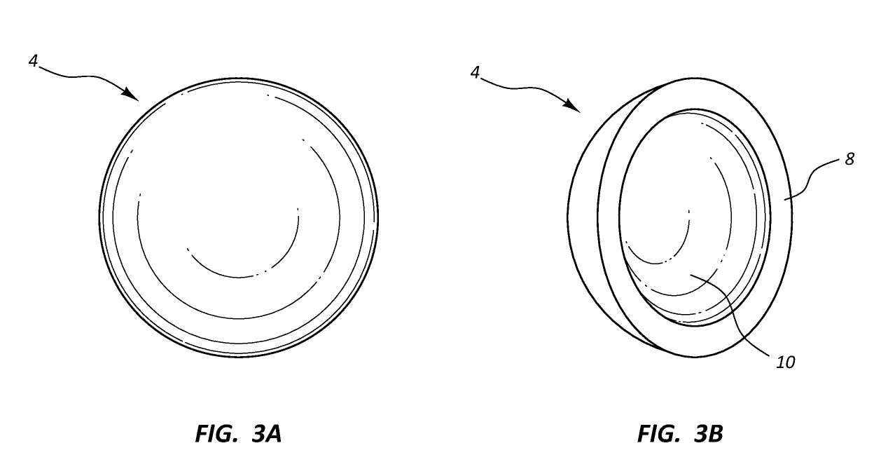

FIGS. 3A and 3B show an exemplary embodiment of a treatment element 4 formed as a treatment ball. The treatment element(s) 4 may be used to apply pressure and/or a desired temperature to an injured portion of a person's body (e.g., an ear). The treatment element(s) 4 may be formed in a variety of shapes and sizes and using a variety of materials. In one embodiment, the treatment element(s) 4 may be disc-shaped, spherical, ovoid, pyramidal, cube-shaped, polygonal, or another shape. In one embodiment, one or more of the treatment elements may be formed as a treatment ball, e.g., similar to the treatment ball shown in FIGS. 1-3. In one embodiment, one or more treatment element(s) 4 may be solid throughout. In one embodiment, as shown for example in FIG. 3B, one or more treatment element(s) 4 may include a shell or outer wall 8. The shell or outer wall 8 may contain/enclose a gas, liquid, gel, foam, solid material, or a combination of materials in an interior space 10 enclosed by the shell or outer wall 8. For example, the shell or outer wall 8 may contain/enclose a cooling gel, a Thermocool gel, a gel or liquid that will freeze solid in a typical freezer (e.g., to form ice), a gel or liquid that will not freeze solid in a typical freezer, or other type of gel or liquid. The treatment element(s) may be formed of one material or a combination of materials. For example, the treatment element(s) 4 and/or outer shell/walls may be formed of polymer materials, polyvinyl chloride (PVC) (e.g., a soft, flexible PVC), rubber, putty, latex, polyurethane, silicone, metal, steel, aluminum, gel, foam, sponge, other materials, or a combination of some or all of these. In one embodiment, the treatment element may be formed of putty or another shapeable material that allows the user to customize the shape. In one embodiment, the treatment element may include a shell or outer wall with an opening, a valve, and/or an otherwise accessible interior such that a user or manufacturer may fill the treatment element with a desired substance (e.g., a gas, liquid, gel, etc.) and/or remove a substance from the interior.

In one embodiment, the treatment element(s) may be flexible or include a shell or outer wall 8 that is flexible. A flexible treatment element or flexible outer shell/wall 8 may allow the treatment element(s) to conform or partially conform to the treatment area (e.g., an injured portion of the ear), or otherwise increase the surface area of the treatment element in contact with the treatment area. Increasing the surface area of contact through conforming or other flexing can allow the treatment element to be used to treat a larger area with the desired pressure and temperature. FIG. 2 shows an exemplary treatment device in a closed or clamped configuration with a lower surface of treatment element/ball 4 compressed against the anvil or platform 24 at a contact region 32, such that the lower surface of the treatment element/ball 4 is flexed, deformed, or flattened against the anvil or platform 24 and a larger surface area of the treatment element/ball 4 contacts the anvil or platform 24 than would if the ball were solid and non-flexible or rigid.

However, rigid or semi-rigid treatment elements may be used for a narrower, more targeted treatment area or other purposes if desired. In one embodiment, the flexural modulus of the treatment element or a shell or outer wall of the treatment element may be between 2 Kpsi and 500 Kpsi, between 5 Kpsi and 100 Kpsi, or another amount. The durometer of the treatment element or a shell or outer wall of the treatment element may be between 1 A and 100 A, between 1 D and 100 D, between 25 D and 75 D, or another amount.

The treatment element(s) may be used to apply a desired temperature to a treatment area (e.g., a portion of a person's body). A temperature may be imparted on one or more of the treatment element(s). For example, the treatment element(s) may be cooled (e.g., in a freezer, refrigerator, etc.) and/or may be heated or warmed (e.g., in an oven, microwave, heater, etc.). The treatment element(s) maybe be configured or designed to retain an imparted temperature (or temperature range) for a period of time, e.g., anywhere from 1-45 minutes, 2-30 minutes, 5-15 minutes, about 10 minutes (±1 minute) or another amount of time. The temperature can increase or decrease over the period of time, but remain below or above room temperature for the period of time. In one embodiment, the treatment elements may be cooled and maintain a temperature(s) cooler than room temperature for at least 10 minutes. The temperature may gradually increase over the 10 minutes, but remains below room temperature for at least 10 minutes. The interior and/or exterior of the treatment element(s) 4 may be designed to maintain the temperature of the treatment element(s) 4 within a temperature range over a period of time. In one embodiment, the exterior of a treatment element 4 (e.g., the shell or outer wall 8) may be constructed of a material that can contain an interior portion 10 (e.g., holding a gel, foam, liquid, etc.) and allow heat energy to transfer between the interior portion and the portion of a person's body being treated (e.g., an area of broken cartilage in a person's ear or other treatment area). The interior portion may include a cooling gel (e.g., a Thermocool gel) that absorbs heat from the treatment area to cool the treatment area.

A treatment system or treatment device may include one or multiple treatment elements of the same or different types (e.g., 1-50, 2-30, 3-15, 12, 6, 3, or another number of treatment elements). The treatment element(s) may be configured and designed to be removable and/or be interchangeable. For example, the treatment ball 4 shown in FIG. 1 may be removable from the upper clamp portion 12 (e.g., from a receptacle 20 therein), and another treatment ball may be inserted in its place for the same or a different purpose. For example, a user may have a group of treatment elements (e.g., 2-10 treatment balls) that are the same or similar and may cool them all (e.g., in the freezer) for application of cold temperatures to a treatment area of a person's body (e.g., an injured area of an ear). The user may add a first cold treatment element to the receptacle 20 and apply treatment to the treatment area for a period of time. Then as the treatment element warms over time, the user may remove the first treatment element and replace it with a second cold treatment element (e.g., taken from the freezer or refrigerator) and so on with multiple or all treatment elements. If different types of treatment elements are used or the treatment elements are at different temperatures, the user may remove one type or temperature of treatment element and replace it with another type or temperature of treatment element for a varied or customized treatment. For example, a user may wish to use a cold temperature treatment element for only a limited period of time to avoid frostbite or other issues but maintain pressure on the treatment area, so after the limited period of time, the user may remove the cold temperature treatment element from the receptacle 20 and replace it with a more moderate temperature (e.g., a room temperature or only slightly cool) treatment element. In one embodiment, a treatment may involve changing/alternating between cold and warm treatment balls and/or between flexible and rigid treatment balls.

Clamping or securement mechanism 6 may be formed in a variety of ways. The clamping/securement mechanism 6 may be configured/designed to apply pressure to a particular treatment area or portion of a person's body. In one embodiment, the clamping/securement mechanism 6 may include two opposable portions, e.g., an upper clamp portion 12 and a lower clamp portion 14, that can be biased, clamped, or secured in relation to each other so as to create a pressure between the two opposable portions. The opposable portions may be directly connected to each other, indirectly connected to each other, attracted to each other by magnetic or other forces, and/or otherwise associated with each other. As shown in FIGS. 1 and 2, the clamping or securement mechanism 6 may be configured as a clamp with two opposable portions hinged together. The ends opposite the hinge may be clamped and maintained at different distances apart, e.g., to create more or less pressure on a treatment area between the opposable portions. In one embodiment, the opposable portions may each include magnets and/or a ferromagnetic material configured such that the opposable portions are biased or attracted toward each other by magnetic force(s). The opposable portions may be physically connected (e.g., by a hinge) or not physically connected (e.g., only paired by magnetic force(s)). If magnets and/or ferromagnetic materials are used, they should be of sufficient strength to attract each other through the treatment area of a person's body (e.g., to attract each other through a portion of a person's ear) and create pressure between the opposable portions on the treatment area via the magnetic attraction/force(s). Other mechanisms for clamping, tightening, attaching, locking, or biasing the opposable portions are also possible, e.g., one or more clamps, ratchets, clasps, catches, locks, snaps, buttons, mating portions, interlocking portions, latches, cinches, ties, pegs, slots, racks and pinions, hooks, pulleys, levers, screws, bolts, treaded regions, a combination of more than one of these, etc. In one embodiment, a headset, headphones, or a construction similar to headphones may be provided with clamping securement mechanism on either end to pinch, clamp, treat, etc. both ears of a person at the same time.

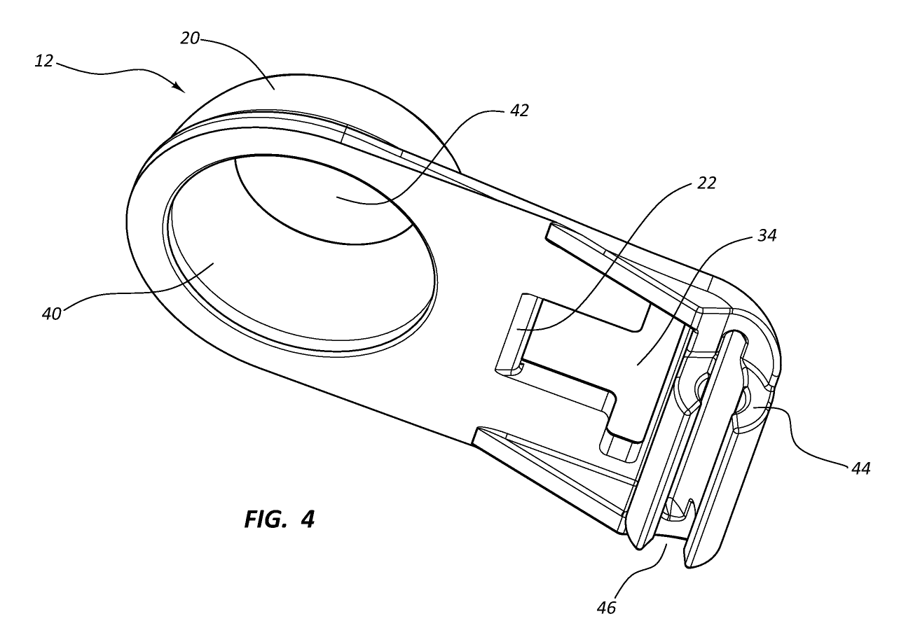

FIG. 4 shows an exemplary embodiment of an upper clamp portion 12. Upper clamp portion 12 may include a receptacle 20 configured/designed to securely hold a treatment element. The receptacle 20 may be configured/designed to allow the treatment element to be removed from the receptacle 20 and the same or a new treatment element replaced in the receptacle 20. The receptacle 20 may include an inner surface 40, and inner surface 40 may be concave, partially concave, or may be otherwise shaped to receive a treatment element or correspond to the shape of a treatment element. The receptacle may be approximately the same size or slightly smaller than the treatment element to provide a friction fit for the treatment element to be held in the receptacle. The receptacle 20 may also include an opening 42 that may allow a portion of the treatment element to be visible or extend from the top of the upper clamp portion 12. Opening 42 may also help facilitate removal or interchangeability of treatment elements in the receptacle 20 by allowing a user to push on the treatment element through the opening 42 to help push it out of the receptacle.

Upper clamp portion 12 may also include one or more mechanisms (e.g., similar to those discussed above) for clamping, tightening, attaching, locking, or biasing the upper clamp portion with another opposable portion, e.g., with lower clamp portion 14. In one embodiment, upper clamp portion 12 may include a hinge or a portion of a hinge that includes a slot or channel 46 defined or encircled by a full or partial wall or sleeve 44. The wall or sleeve 44 may be configured in a columnar or partially columnar shape and may have a closed or open circumference. The slot or channel 46 may be shaped, designed, and configured to receive another component 16 (e.g., a post, pin, peg, bolt, portion of an opposable portion or lower clamp portion, or other component). The hinge or portion of the hinge may be configured and designed such that the wall or sleeve 44 can rotate around the component received in the slot or channel 46 so a hinge or pivot point may be created. In one embodiment, the lower clamp portion 14 may include one or more components (e.g., bars, pegs, pins, posts, bolts, etc.) that fit within the slot or channel 46 to form a hinged connection with the upper clamp portion 12. In one embodiment, the lower clamp portion 14 may include one or more of its own holes, slots, or channels 48 (which may, for example, be formed by a full or partial wall or sleeve) that may be fitted with the upper clamp portion such that the holes/slots/channels of each are aligned and a component/connector 16 (e.g., a post, pin, peg, bolt, etc.) may be passed through the slots/channels of each to form a hinged connection between the lower clamp portion 14 and the upper clamp portion 12 (e.g., similar to a door hinge that has channels on each side and a pin or bolt that passes through the channels to connect the hinge).

Upper clamp portion 12 may also include an opening or slot 34 configured or shaped to receive and/or surround a portion of a lock/release lever 18. Upper clamp portion 12 may also include an edge or mating portion 22 that may interact or mate with one or more teeth 28 on lock/release lever 18 to hold the upper clamp portion 12 in a desired position or configuration relative to the lock/release lever 18 and relative to the lower clamp portion 14.

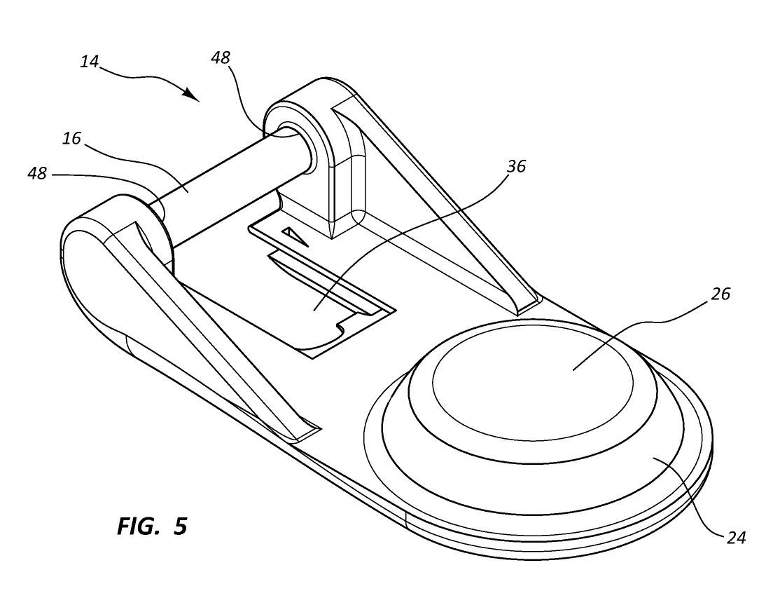

FIG. 5 shows an exemplary embodiment of a lower clamp portion 14. Lower clamp portion 12 may include an anvil or platform 24 against which pressure may be applied (e.g., by the treatment element). The anvil or platform 24 may include a contact surface 26 that contacts the treatment element or a treatment area of a person's body (e.g., when the treatment area is clamped between the treatment element and the anvil or platform 24). The anvil or platform 24 and the contact surface 26 can be a variety of shapes (e.g., round, hemispherical, plateau-like, flat, concave, convex, a shape that conforms to an ear or other portion of a body) and sizes (e.g., smaller for more targeted treatment or larger for broader treatment/coverage). A flat contact surface 26 may help a flexible treatment element to conform or flatten to have a larger surface area in contact with the anvil or platform 24 or in contact with a treatment area of a person's body (e.g., an ear).

Optionally, lower clamp portion 14 may include a receptacle (e.g., the same as or similar to receptacle 20 of the upper clamp portion 12) configured/designed to securely hold a treatment element. The receptacle may be configured/designed to allow the treatment element to be removed from the receptacle and the same or a new treatment element replaced in the receptacle. The receptacle may include an inner surface (e.g., the same as or similar to inner surface 40 discussed above), and the inner surface may be concave, partially concave, or may be otherwise shaped to receive a treatment element or correspond to the shape of a treatment element. The receptacle may also include an opening (e.g., the same as or similar to opening 42 discussed above) that may allow a portion of the treatment element to be visible or extend from the bottom of the lower clamp portion 14. The opening may also help facilitate removal or interchangeability of treatment elements in the receptacle by allowing a user to push on the treatment element through the opening to help push it out of the receptacle. If both the upper clamp portion 12 and the lower clamp portion 14 include a receptacle for receiving a treatment element, the clamp or clamping/securement mechanism 6 may clamp or close such that a treatment element in each receptacle is pressed against one another or against opposite sides of a treatment area of a person's body. A flexible treatment element on either side of the treatment area may allow the treatment device to better conform or shape itself for optimal treatment of the treatment area. Also, a treatment element on either side of the treatment area may allow the application of desired temperatures to either side of the treatment area (e.g., cold or cool on both sides, hot or warm on both sides, or hot/warm on one side and cold/cool on the other side).

Lower clamp portion 14 may also include one or more mechanisms (e.g., similar to those discussed above) for clamping, tightening, attaching, locking, or biasing the lower clamp portion with another opposable portion, e.g., with upper clamp portion 12. In one embodiment, lower clamp portion 14 may include a hinge or a portion of a hinge that includes one or more holes, slots, or channels 48, which may be defined or encircled by a full or partial wall or sleeve. The wall or sleeve may be configured in a columnar or partially columnar shape and may have a closed or open circumference. The holes, slots, or channels 48 may be shaped, designed, and configured to receive another component 16 (e.g., a post, pin, peg, bolt, portion of an opposable portion or upper clamp portion, or other component). The hinge or portion of the hinge may be configured and designed such that the wall or sleeve can rotate around the component received in the hole, slot, or channel 48 so a hinge or pivot point may be created. Lower clamp portion 14 may also include an opening or slot 36 configured or shaped to receive and/or surround a portion of a lock/release lever 18.

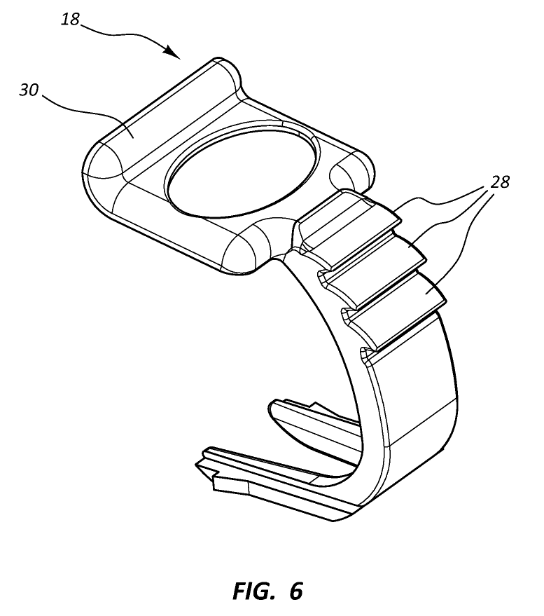

The clamp/securement mechanism 6 may include a lock/release lever 18. FIG. 6 shows an exemplary embodiment of a Lock/release lever 18 that may be used with the clamp/securement mechanism 6. Lock/release lever 18 may include one or more teeth 28 and a gripping feature 30. Lock/release lever 18 may also include a logo or other information in one or more locations, e.g., on the oval portion near the gripping feature 30 in FIG. 6. The teeth 28 may mate or otherwise interact with an edge or mating portion 22 of a clamp/securement mechanism 6. The edge or mating portion 22 may fit between teeth 28 or below one of the teeth 28 to hold or lock the clamp/securement mechanism 6 in a closed or clamped configuration. Different closed or clamped configurations and levels of pressure between the opposable portions may be possible because of multiple teeth 28 (e.g., lower teeth resulting in a tighter or smaller configuration and more pressure between opposable portions). Gripping feature 30 may help a user to grasp and move or otherwise push the lock/release lever 18 in a desired direction to lock the clamp/securement mechanism 6 in a closed or clamped configuration or to release the clamp/securement mechanism 6 from the closed or clamped configuration and return it to an open configuration. To release the clamp/securement mechanism 6 from the closed or clamped configuration and return it to an open configuration, a user may push or pull or otherwise apply a force to the lock/release lever 18 such that the teeth 28 are moved in a direction away from the edge or mating portion 22 and disengage from the edge or mating portion 22. This frees the upper clamp portion 12 and the lower clamp portion 14 to move apart and return to an open configuration.

While FIGS. 1 & 2 show the clamping/securement mechanism configured as a clamp that holds only one treatment element and applies pressure between the treatment element and the anvil or platform 24, other clamping/securement mechanisms are possible. In one embodiment, the upper clamp portion 12 and the lower clamp portion 14 may be of similar construction or may both include a receptacle portion 20 for receiving a treatment element 4. For example, instead of anvil or platform 24, the lower clamp portion 14 may include another receptacle (e.g., similar to receptacle 20) for receiving another treatment element 4. The treatment elements 4 could be of a variety of configurations and used for a variety of purposes as discussed in more detail above. In one embodiment, two rigid, two flexible, and/or one flexible and one rigid element(s) might be used. A treatment element that forms an anvil or platform similar to anvil or platform 24 may be used, but could be removable and replaced with a different treatment element if desired. In one embodiment, two cool/cold, two warm/hot, and/or one cool/cold and one warm/hot element(s) might be used. In one embodiment, a treatment element including or constructed of conformable putty or a similar conformable material may be used as one or more of the treatment elements.

A treatment system may include treatment device 2, any of its components/features, and/or any other additional treatment items desired. In one embodiment, a treatment system may include treatment device 2, including a clamp or clamping/securement mechanism and one or more treatment elements. The treatment system may also include additional devices or components for use after treatment with treatment device 2, e.g., a lotion, brace, cover, or something else.

The apparatuses, devices, systems, components, etc. described herein may be manufactured in a variety of ways, including using a mold or cast, injection molding, 3D printing, welding, carving, other methods, and/or a combination of these. All or some materials used to make the apparatuses, devices, systems, components, etc. discussed above may be polymer materials, polyurethane, silicone, latex, PVC, PTFE, wood, metals, rubber, putty, latex, steel, aluminum, gel, foam, sponge, other materials, or a combination of some or all of these. Methods of manufacturing may include taking steps to form and/or combine/assemble components or features described in the order/arrangement shown or described herein.

While generally described herein in terms of a treatment device or ear treatment device, the principles and design features described may also be used with different types of treatment devices, medical devices, any other devices requiring the simultaneous application of pressure and a desired temperature, etc.

The treatment devices and components thereof described herein may be used in a variety of methods. Methods of using the treatment devices or any of their components may include any of the steps discussed anywhere in this description or the summary, including steps of (1) providing or obtaining a treatment device (e.g., having some or all of the features of treatment device 2 described herein), (2) selecting one or more desired treatment element(s) for treatment of a treatment area of a person's body (e.g., an injured portion of an ear), (3) preparing the treatment element(s) for treatment (e.g., cooling the treatment element to a desired temperature, heating the treatment element to a desired temperature, shaping the treatment element into a desired shape, etc.), (4) fitting, placing, securing, attaching, etc. one or more treatment element(s) (e.g., which may be the same as or similar to treatment element(s) 4 as described herein) in one or more receptacle(s) (e.g., which may be the same as or similar to receptacle 20) of the treatment device and/or a clamp or clamping/securement mechanism or portion thereof, (5) positioning the treatment device in a desired location (e.g., at or near a treatment area of person's body), (6) clamping, attaching, securing, etc. the treatment device at or near the treatment area (e.g., such that the treatment element(s) applies pressure directly or indirectly to the treatment area), (7) applying pressure and/or a desired temperature to the treatment area with the treatment element(s) and treatment device for a period of time (e.g., applying a cold temperature and pressure via the treatment element for 5-10 minutes), and (8) unclamping, detaching, removing, etc. the treatment device from the treatment area after the period of time. Any combination of one or more of these steps might be taken. Additional steps might also be taken including, for example, (9) detaching, removing, etc. the treatment element(s) from the receptacle(s) of the treatment device, (10) selecting and/or preparing the same or different treatment element(s) for additional treatment (e.g., cooling the treatment element to a desired temperature, heating the treatment element to a desired temperature, shaping the treatment element into a desired shape, etc.), (11) fitting, placing, securing, attaching, etc. one or more treatment element(s) in one or more receptacle(s) of the treatment device and/or a clamp or clamping/securement mechanism or portion thereof, (12) repeating any or all of steps 5-11. These and other steps described herein may be performed in the order disclosed or in a different sequence, and steps may be omitted or added.

The above apparatuses, devices, components, systems, assemblies, methods, etc. have generally been described and shown in figures as being applied to ear treatment devices and their components; however, the principles described may be applied to a variety of other apparatuses, devices, components, systems, assemblies, methods, etc. Further, the features described in one embodiment herein may generally be combined with features described in other embodiments herein.

While the apparatuses, devices, components, systems, assemblies, methods, etc. of this invention may have been described in terms of particular variations and illustrative figures, it will be apparent to those skilled in the art that the invention is not so limited and that variations may be applied to other shelters, apparatuses, devices, components, systems, assemblies, methods, etc. For example, with respect to the methods, uses, and/or steps described herein variations may occur in the steps, uses, the sequence/order of steps, etc. described herein without departing from the concept, spirit, and scope of the invention, as defined by the claims. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A treatment system for treating a treatment area of a person's body, comprising:
a first treatment element; and
a securement mechanism comprising a first opposable portion and a second opposable portion, the first opposable portion including a first receptacle configured to releasably hold the treatment element, the securement mechanism designed to transition between an open configuration and a closed configuration, wherein a distance between the first opposable portion and the second opposable portion is greater in the open configuration than in the closed configuration.

2. The treatment system according to claim 1, wherein the first treatment element is a treatment ball.

3. The treatment system according to claim 1, wherein the first treatment element comprises an outer shell and includes at least one of a liquid and gel inside the outer shell.

4. The treatment system according to claim 3, wherein the first treatment element is designed to be cooled to a temperature below room temperature and remain below room temperature for a period of time longer than 4 minutes.

5. The treatment system according to claim 1, wherein the first treatment element is designed to be cooled to a temperature below room temperature and remain below room temperature for a period of time longer than 8 minutes.

6. The treatment system according to claim 1, wherein the first treatment element includes an outer surface and has a flexibility to change a shape of the outer surface when pressure is applied to the treatment area with the first treatment element.

7. The treatment system according to claim 1, wherein the second opposable portion includes a platform configured to apply an opposing force to the first treatment element in the closed configuration.

8. The treatment system according to claim 7, wherein the second opposable portion includes a second receptacle capable of receiving a second treatment element.

9. The treatment system according to claim 1, wherein the securement mechanism is configured such that the distance between the first opposable portion and the second opposable portion can be adjusted between three closed configurations that each apply a different amount of pressure to the treatment area.

10. The treatment system according to claim 1, wherein the treatment system is an ear treatment system.

11. A method comprising:
obtaining a treatment device for treating a treatment area of a person's body, the treatment device comprising:
a first treatment element; and
a securement mechanism comprising a first opposable portion and a second opposable portion, the first opposable portion including a first receptacle configured to releasably hold the treatment element, the securement mechanism designed to transition between an open configuration and a closed configuration;
altering a temperature of the first treatment element from a first temperature to a second temperature different from the first temperature;
placing the first treatment element in the receptacle such that it is held by the first opposable portion;
securing the treatment device at the treatment area by transitioning the treatment device from the open configuration to the closed configuration such that, in the closed configuration, the first treatment element applies pressure to the treatment area; and
removing the treatment device from the treatment area.

12. The method according to claim 11, further comprising applying pressure to the treatment area with the first treatment element for a period of time between 2-15 minutes before removing the treatment device from the treatment area.

13. The method according to claim 11, further comprising removing the first treatment element from the receptacle.

14. The method according to claim 13, further comprising altering a third temperature of the first treatment element to a fourth temperature of the treatment element, the fourth temperature being different from the third temperature, then returning the first treatment element to the receptacle such that it is held by the first opposable portion.

15. The method according to claim 14, further comprising resecuring the treatment device at the treatment area such that the first treatment element applies pressure to the treatment area.

16. The method according to claim 13, further comprising obtaining a second treatment element different from the first treatment element, and placing the second treatment element in the receptacle such that it is held by the first opposable portion.

17. The method according to claim 16, further comprising resecuring the treatment device at the treatment area such that the second treatment element applies pressure to the treatment area.

18. The method according to claim 11, wherein the second temperature is colder than the first temperature, and wherein altering the temperature of the first treatment element from the first temperature to the second temperature includes cooling the first treatment element in at least one of a refrigerator or a freezer.

19. The method according to claim 11, wherein the securement mechanism comprises a lock/release lever that has teeth, and wherein securing the treatment device at the treatment area includes adjusting a distance between the first opposable portion and the second opposable portion until the teeth lock the first opposable portion and the second opposable portion in a first position that applies a first pressure to the treatment area.

20. The method according to claim 19, wherein securing the treatment device at the treatment area further includes readjusting the distance between the first opposable portion and the second opposable portion until the teeth lock the first opposable portion and the second opposable portion in a second position that applies a second pressure to the treatment area, wherein the second pressure is different from the first pressure.

21. The method according to claim 19, wherein removing the treatment device from the treatment area includes adjusting the lock/release lever such that the first opposable portion and the second opposable portion are released from the first position and the treatment device is able to transition to the open configuration.

22. The method according to claim 11, wherein the treatment area is a portion of an ear.
